(19) United States
(12) Patent Application Publication
(10) Pub. No.: US 2012/0150306 A1
(43) Pub. Date: Jun. 14, 2012

(54) HINGE STRUCTURE

(76) Inventor: Roelof Marissen, Born (NL)

(21) Appl. No.: 13/376,459

(22) PCT Filed: Jun. 15, 2010
(86) PCT No.: PCT/EP2010/058354

§ 371 Date: Feb. 29, 2012

(30) Foreign Application Priority Data
Jun. 15, 2009 (EP) 09162696.0

(51) Int. Cl. A61F 2/40; A61F 2/32; E05D 5/02

(52) U.S. Cl. 623/19.13; 16/385; 623/22.15

(57) ABSTRACT

The invention relates to a three-dimensional hinge (100) comprising a first rigid body (10), a second rigid body (20) and a joint member. The joint member connects the first rigid body (20) and the second rigid body (20). The joint member comprises at least three base connectors (32,33,34), each base connector connected to the second rigid body (20) at respective connection point (22, 23, 24) and engaging with an engaging portion of the first rigid body.

The invention relates to a hinge. The invention also relates to devices comprising such a hinge, such as an apparatus or a medical device comprising such a hinge, in particular to an artificial hip joint comprising such a hinge.

Medical products such as artificial joints typically utilize a combination of polymer and metallic alloys. The metallic alloys are employed for the articulation and the polymer is employed as the soft socket. The polymer part wears against the metal articulation part during use, and ultrafine particles of polymer are loosened into the body over time. These particles are known to cause resorption of the periprosthetic bone. This results in loosening of the prosthesis components, possibly requiring even revision surgery.

U.S. Pat. No. 5,549,700 discloses a ball and socket prosthetic joint device. The socket component includes a polymeric cup insert and a plurality of bearing segments embedded therein. The femoral component includes a joint motion surface in the form of a ball element. The femoral ball element has a spherical exterior bearing surface which is maintained in slidable contact with the bearing surfaces of the segments. Although the plurality of bearing segments results in lower stress being transferred between contacting bearing surfaces than a construction without such bearing segments, the problem of polymer loosening into the body is still not solved.

The object of the present invention is therefore to provide an improved hinge in which the problem is reduced.

The object is achieved according to the invention by a hinge comprising a first rigid body, a second rigid body and a joint member connected to the first rigid body and the second rigid body. The joint member comprises at least three base connectors, each base connector connected to the second rigid body at respective connection point and engaging with an engaging portion of the first rigid body, wherein the base connectors are yarns made from fibers and at least a part of the fibers are synthetic organic fibers. The first rigid body can move with respect to the second rigid body, having the engaging portion of the first rigid body as the fulcrum. When the second rigid body is pushed, pulled or rotated with respect to the first rigid body, the yarns stop the movement at a certain degree. The degree of the movement depends on how tight the yarns are tensioned between the two bodies, the angles between the yarns and the stiffness of the yarns.

A major advantage of the present invention is that the use of yarns as base connectors. The yarns are stiff under tension but flexible under compression by torsion or linear pressure, which allows the hinge to carry even a large load while maintaining ability to move (for example tilt and/or twist) with no or only very limited friction in the system as there is no direct sliding contact between the first and the second body.

The number of the base connectors in the joint member is at least three. In some cases, it may be advantageous that the number of the base connectors is four, five, six or even more from higher mechanical strength point of view or better control of forces or range of motion. However, the number of the base connectors is preferably three. This is advantageous in that small amount of materials is used and also that internal stresses caused by the tension or torsion from the base connectors can be minimized. When the joint member comprises five or more base connectors, differences in the length of the yarns may result in different forces being applied to different yarns causing internal stress but also a way to better control range of motion.

By this invention, a hinge is provided in which two rigid bodies are connected but do not make an abrasive sliding motion with respect to each other over the surfaces thereof. This is extremely important in that there is no wear between the first body and the surface of the second body facing the first body upon movement of the bodies relative to each other, which means that there will be no or only limited abrasion between the rigid bodies. When the first rigid body is tilted with respect to the second rigid body, the degree of the tilt may be limited by the first rigid body touching upon the rim of the surface of the second rigid body facing the first body. Rotating the first rigid body in that tilted position results in the first rigid body sliding against the rim. However, the effect caused by this sliding movement is substantially smaller than the abrasion caused by the rotational movement in a construction where a ball slides against a cup, such as in conventional artificial hip joints. According to the hinge of the present invention, abrasion occurs only when the first rigid body moves at a certain extremely tilted position with respect to the second rigid body, whereas in conventional artificial hip joints abrasion takes place with any range of motion.

The rim of the surface of the second rigid body facing the first rigid body and/or the part of the first rigid body which may come into contact with the rim may be made to have an improved anti-abrasive property. This may be done by e.g. providing an anti-abrasive coating at these parts.

Accordingly, the first and the second rigid bodies may be made of any materials, including metal, ceramic and polymer and a combination thereof, with substantially reduced risk of abrasive damage in the rigid bodies. Metal is preferred. In case of metal, a surgical stainless steel or a titanium alloy is preferred for use in medical applications.

Preferably, the hinge according to the present invention is a three-dimensional hinge. A three-dimensional hinge is herein meant a hinge which allows two rigid bodies connected to each other by the hinge itself, to move relative to each other allowing a rotation within a fixed solid angle relative to each other.

In a highly preferred embodiment, the hinge comprises a further connector connected to the second rigid body at a further connection point and engaging with an engaging portion of the first rigid body. The further connecting point is arranged in such a way that the connection points of the base connectors and the further connection point define the corners of an (imaginary) polyhedron within which the engaging portion of the first rigid body is positioned. When the number of the base connectors is four, the polyhedron is a tetrahedron. The fact that the engaging portion of the first rigid body is positioned within the polyhedron ensures that the engaging portion of the first rigid body is tensioned in at least four directions. This allows the engaging portion of the first rigid body to be maintained inside the polyhedron without contacting or moving through its imaginary faces.

The two rigid bodies may be connected in such a way that the relative movement between the first rigid body and the second rigid body is limited by the base connectors so that the two bodies do not contact each other. This can be done by suitably choosing the connection points of the connectors to the first rigid body and the properties of the base connectors, such as for example thickness, material, arrangement of fibers in the yarn. This is extremely advantageous in that no damage by abrasion occurs between the two rigid bodies. Another advantage of such a hinge where no contact occurs is that no electrical connection is made between the two rigid bodies if a non-conducting yarn is chosen. This may for example be advantageous where the hinge is used in robot applications, such as in a robot arm where electrical contact damages it.

By a yarn is herein meant any elongated body with its transversal dimension much smaller than its length made from fibers by twisting, twining, braiding or the like, and includes a cable or a braid. The yarn may comprise a further element, such as a coating, a cover or a (cured) resin. At least a part of the fibers are synthetic organic fibers, such as for example polyester based, polyamide based, polyurethane based, polyethylene based.

Preferably, each of the base connector yarns and/or the further connector yarn comprises high performance polyethylene (hereinafter HPPE) fibers. The HPPE fibers in the yarns provide an extremely strong connection between the first and the second rigid bodies. The tenacity of the HPPE fibers in the joint member prevents the breakage of the HPPE fibers and allows for the use of relatively thin yarns hence opening new application via miniaturized hinge design. The HPPE fibers also show a very high resistance against bend fatigue. This is extremely advantageous since the most important function of a hinge is to be able to be bent numerous times without breaking.

Within the context of the present invention, fibres are understood to mean elongated bodies of virtually indefinite length and with length dimension much greater than width and thickness. The term fibre thus includes a monofilament, a multifilament yarn, a ribbon, a strip or tape and the like, and can have regular or irregular cross-section. The term fibres also includes a plurality of any one or combination of the above.

Fibres having the form of monofilaments or tape-like fibres can be of varying titer, but typically have a titer in the range of 10 to several hundred thousand dtex, preferably in the range of 100 to 25000 dtex, more preferably 200-10000 dtex. Multi-filament yarns contain a plurality of filaments having a titer typically in the 0.2-25 dtex range, preferably about 0.4 or 0.5 to 20 dtex. The titer of a multifilament yarn may also vary widely, for example from 10 or 15 to several hundred thousand dtex, but is preferably in the range of about 100-20000 dtex, more preferably 300-10000 dtex.

HPPE fibres are herein understood to be fibres made from polyethylene and having a tenacity of at least 1.4, preferably at least 2.0, more preferably at least 2.5 or even at least 3.0 N/tex. Tensile strength, also simply strength, or tenacity of fibres are determined by known methods, as based on ASTM D885-85 or D2256-97. There is no reason for an upper limit of tenacity of HPPE fibres, but available fibres typically are of tenacity at most about 5 to 6 N/tex. The HPPE fibres also have a high tensile modulus, e.g. of at least 50 N/tex, preferably at least 75, at least 100 or at least 125 N/tex. HPPE fibres are also referred to as high-modulus polyethylene fibres.

HPPE fibres can be prepared by spinning of a solution of ultrahigh molecular weight polyethylene (UHMWPE) in a suitable solvent into gel fibres and drawing the fibres before, during and/or after partial or complete removal of the solvent; that is via a so-called gel-spinning process. Gel spinning of a solution of UHMWPE is well known to the skilled person; and is described in numerous publications, including EP 0205960 A, EP 0213208 A1, U.S. Pat. No. 4,413,110, GB 2042414 A, EP 0200547 B1, EP 0472114 B1, WO 01/73173 A1, and in Advanced Fiber Spinning Technology, Ed. T. Nakajima, Woodhead Publ. Ltd (1994), ISBN 1-855-73182-7, and in references cited therein, all incorporated herein by reference.

UHMWPE is understood to be polyethylene having an intrinsic viscosity (IV, as measured on solution in decalin at 135° C.) of at least 5 dl/g, preferably of between about 8 and 40 dl/g. Intrinsic viscosity is a measure for molar mass (also called molecular weight) that can more easily be determined than actual molar mass parameters like Mₙ and M_{w}. There are several empirical relations between IV and M_{w}, but such relation is dependent on molar mass distribution. Based on the equation M_{w}=5.37*10⁴ [IV]^{1.37} (see EP 0504954 A1) an IV of 8 dl/g would be equivalent to M_{w} of about 930 kg/mol. Preferably, the UHMWPE is a linear polyethylene with less than one branch per 100 carbon atoms, and preferably less than one branch per 300 carbon atoms; a branch or side chain or chain branch usually containing at least 10 carbon atoms. The linear polyethylene may further contain up to 5 mol % of one or more comonomers, such as alkenes like propylene, butene, pentene, 4-methylpentene or octene.

When HPPE fibers are prepared by the gel-spinning process, the residual solvent content is preferably at most 100 ppm. This is especially important when the hinge is used in medical applications. The residual solvent content is herein understood to mean the content of the solvent used in making the HPPE fibers, which is still remaining in the final fibers. In the process of making the yarn, any of the known solvents for gel spinning of UHMwPE can be used.

HPPE fibers can also be prepared by melt-spinning of polyethylene having a high molecular weight, although the mechanical properties such as tenacity are more limited compared to HPPE fibres made by the gel-spinning process. The upper limit of the molecular weight of the polyethylene which can be melt-spun can be in the range of the molecular weight of UHMWPE, but is lower than the limit with the gel-spinning process. Melt-spun HPPE fibers can also be prepared from polyethylene having a molecular weight lower than UHMWPE as defined above. The melt-spinning process is widely known in the art, and involves heating a PE composition to form a PE melt, extruding the PE melt, cooling the extruded melt to obtain a solidified PE, and drawing the solidified PE at least once. The process is mentioned e.g. in EP1445356A1 and EP1743659A1, which are incorporated herein by reference.

In one embodiment, the UHMWPE contains a small amount, preferably at least 0.2, or at least 0.3 per 1000 carbon atoms, of relatively small groups as pending side groups, preferably a C1-C4 alkyl group. Such a fibre shows an advantageous combination of high strength and creep resistance. Too large a side group, or too high an amount of side groups, however, negatively affects the process of making fibres. For this reason, the UHMWPE preferably contains methyl or ethyl side groups, more preferably methyl side groups. The amount of side groups is preferably at most 20, more preferably at most 10, 5 or at most 3 per 1000 carbon atoms.

The HPPE fibres may further contain small amounts, generally less than 5 mass %, preferably less than 3 mass % of customary additives, such as anti-oxidants, thermal stabilizers, colorants, flow promoters, etc. The UHMWPE can be a single polymer grade, but also a mixture of two or more different polyethylene grades, e.g. differing in IV or molar mass distribution, and/or type and number of co-monomers or side groups.

Preferably, the first and/or the second rigid bodies are metals. Metals are preferred because of their tensile strength and compression strength. Alternatively, the first rigid body and/or the second rigid body comprise a composite material including reinforcing fibers impregnated with a resin. The reinforcing fibers may be, for example, carbon, glass, SiC, B, Al₂O₃. Resin is preferably chosen from the biocompatible type of resins.

The second rigid body may be of any shape as long as it has at least one set of three connection points preferably defining a plane. More preferably, the second rigid body also has a further connection point arranged in a way that the connection points and further connection point define a (polyhedron) space. The second rigid body preferably has a concave surface. For example, the second rigid body may have a rectangular cross section, triangular cross section and other shapes. Most preferably, however, the second rigid body has a generally hemispherical shape.

In one embodiment, the second rigid body is formed by at least two segments arranged rigidly relative to each other. This may for example be realized by having the connection points arranged on a plane member (like for example a ring) and the further connection point arranged away from the plane member but connected for example via a bone part to the plane member.

The first rigid body preferably comprises a rod-shaped portion which includes the engaging portion engaging with the base connectors and the optional further connector. In one embodiment, a further connector extends from the engaging portion to its connection point in the second rigid body generally in the direction of the axis of the rod-shaped portion in an unloaded state. By the term “unloaded state”, it is herein meant that no external force is applied to the first and the second rigid bodies to move them with respect to each other. In the unloaded state of the hinge, the base connectors are typically straight between the first and the second body with a certain minimum tension. Hence it should be observed that the so-called unloaded state of the hinge does not refer to the base connectors being un-tensioned, but rather that the tension in the base connectors is balanced.

The further connector limits the movement between the first and the second rigid bodies when they are pulled from each other. In a preferred embodiment, the further connector is of the same type as the base connector, that is a yarn made from fibers and at least a part of the fibers are synthetic organic fibers. The further connector may have substantially the same properties as the base connectors with regard to for example stiffness, strength etc., but in a highly preferred embodiment, the stiffness under tension of the further connector is lower than the stiffness of the base connectors. This allows for the further connector to deform in response to the increased tension of the connectors during twisting and/or turning of the hinge. More preferably, the stiffness of the further connector is less than 50% of the stiffness of the base connectors. The reduced stiffness may for example be realized by a utilizing another type of yarn or another titer of the same yarn. Alternatively, the further connector may be made from a different type of material, like for example an elastomer, such as a rubber or a metal. In this embodiment, the further connector is not a yarn but rather a beam or a spring.

The base connectors preferably extend in the direction which limits the movement between the first and the second rigid bodies when the first and the second rigid bodies are pushed towards each other. The angles between the base connectors are preferably substantially equal in an unloaded state. For example, in cases where the number of the base connectors is three, preferably the triangle defined by the connection points of the base connectors is substantially a regular triangle. In cases where the number of the base connectors is four, preferably the quadrangle defined by the connection points of the base connectors is substantially a square.

Preferably, the angles between any two of the base connectors the further connector are substantially equal in an unloaded state. This means that the angle between the yarns is in the range between about 100° to about 120°, and preferably about 109.5° in case where the number of the base connectors is three.

The yarn of the base connectors and the optional further connector may be attached to the first and the second rigid body in various ways. For example, the rigid body may be provided with a hole through which the yarn penetrates, and a knot is made to prevent the yarn from being removed from the rigid body. For example, by making a knot larger than the diameter of the hole after the yarn penetrated through the hole, the yarn is prevented from being removed from the rigid body with the hole. In one embodiment, the first rigid body is hollow. The yarns penetrate through a hole or holes provided in the first rigid body, and a knot is made inside the hollow body. The knot may be made from one yarn or several yarns. In another embodiment, the first rigid body comprise a protrusion around which a knot is formed. In another embodiment, the first rigid body comprises a ring at its end portion facing the second rigid body and the yarns are wound around the ring. In another embodiment, two or more of the base connectors and the optional further connector may be formed by one yarn running for example from a connection point on the second rigid body via the first rigid body and to the same or another connection point on the second rigid body. This embodiment is highly advantageous as the number of yarn fixations is limited. The present invention also relates to a medical device comprising the hinge according to the present invention. The medical device may be a medical tool or a medical implant. Preferably, the yarns in the joint member substantially consist of HPPE fibers. Preferably, the first and the second rigid bodies substantially consist of biocompatible materials, such as a surgical stainless steel or a titanium alloy, or a polymer, preferably a polyolefin, more preferably polyethylene, most preferably UHMWPE.

The present invention also relates to an artificial joint comprising the hinge according to the present invention.

The present invention also relates to an artificial hip joint comprising the hinge according to the present invention. The artificial hip joint comprises a femur head part and an acetabulum part. The femur head part comprises the first rigid body and the acetabulum part comprises the second rigid body. Due to the presence of the joint member comprising at least four yarns, the femur head part is connected to the acetabulum part, but is never in contact therewith. The problem of polymer particles loosening into the body by abrasion between the femur head part and the acetabulum part is not encountered.

Similarly, the present invention also relates to an artificial shoulder join comprising the hinge according to the present invention. The artificial shoulder joint comprises a humerus head part and a glenoid part. The humerus head part comprises the first rigid body and the glenoid part comprises the second rigid body.

Another aspect of the invention concerns an apparatus comprising the hinge according to the first aspect of the invention. For example, the hinge according to the present invention can be used for various mechanical applications involving repeated bending. Here, the lack of sliding parts leads to very smooth twisting or bending movement of the connected parts with virtually no change in movement pattern over time as typically observed in conventional hinges when friction gradually wears the sliding surfaces of the connected parts. Replacement of ball bearings for applications with smaller alternating forward and back rotations is another major application of this invention. For example, a robot arm comprising the hinge according to the present invention is especially advantageous as well as applications in doors and actuators.

The hinge according to the present invention can also be of an extremely large size. One such example is a dam, such as the dam in the Netherlands called “Maaslandkering”. In Maaslandkering, the gate is opened and closed by a sliding movement of two walls, and the sliding movement is governed by a very large ball-bearing. This ball-bearing may be replaced by the hinge according to the present invention, especially when UHMWPE yarns with a titer in the degree of thousands are used.

The invention is hereinafter further illustrated with reference to the attached drawing in which:

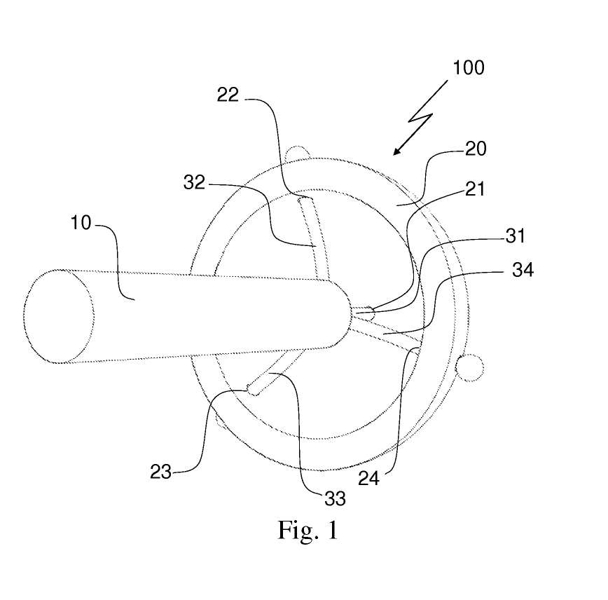

FIG. 1 is a diagrammatic perspective view of an embodiment of the hinge of the present invention.

It is noted that the same reference numbers have been used for corresponding elements in the embodiments.

Referring to FIG. 1, a perspective view of an embodiment of a hinge 100 according to the present invention is shown. The hinge 100 comprises a first rigid body 10 and a second rigid body 20. Here, the first rigid body 10 has a rod shape having an engaging portion at its end portion facing the second rigid body 20, but other shapes of the first rigid member, like for example a (partial) ball shape, a cone shape or an irregular shape is also feasible without departing from the invention. The second rigid body 20 has a hemispherical shape, but other shapes of the second rigid member is also feasible without departing from the invention. Here, the first rigid body 10 extends inside the space surrounded by the concave surface of the second rigid body 20. Four yarns 31, 32, 33, 34 collectively referred as a joint member 30 connects the first and the second rigid body 10 and 20. The base connector yarns 32, 33 and 34 each extends from the engaging portion to connection points 22, 23, 24 of the second rigid body 20. The further connector yarn 31 (which is not an essential feature of the invention but included in this highly preferred embodiment) extends from the engaging portion of the first rigid body 10 to the further connection point 21 of the second rigid body 20 in a generally same direction as the axis of the rod. This allows for a preferred balancing of the forces involved, but arrangement of the further connection point away from the generally same direction as the axis of the rod is also feasible. In some cases—for example with the hinge is not utilized in a substantially vertical arrangement—arrangement of the further connection point 21 away from the generally same direction as the axis of the rod may be highly advantageous. The engaging portion is not shown in FIG. 1, but it may for example be shaped as a loop to which the yarns 31,32,33,34 are connected or as an opening if the first rigid body is hollow. The connection points 22,23,24 and the further connection point 21 define a tetrahedron. With more base connectors higher polyhedrons would be defined. The angle between the yarns is about 109.5 degrees. The engaging portion of the first rigid body 10 is generally at the center of this tetrahedron. Pushing the first rigid body 10 into the concave surface of the second rigid body 20 is not resisted by the further connector yarn 31, but is stopped by the combined effect of the base connector yarns 32,33,34 pushing back the engaging portion in the opposite direction. Pulling the first rigid body 10 away from the second rigid body 20 is resisted by the further connector yarn 31 pulling the engaging portion in the opposite direction. However, if pulling the first rigid body 10 away from the second rigid body 20 is not anticipated by the application, then the further connector is not be required and may be omitted. Turning the first rigid body 10 around its axis with respect to the second rigid body 20 is allowed to a certain extent mainly by the presence of the base connector yarns 32,33,34.

It will be appreciated that various modification to this embodiment is possible. In particular, the connection of the yarns with the first rigid body may be made in many different ways. Different connections may be made as long as the yarns engage with the engaging portion in such a way that the movement is limited within a fixed solid angle when force is applied to the first and the second rigid body. For example, the yarns may penetrate into the first rigid body in shape of a hollow rod and out through holes on its side wall. A knot formed outside the side wall secures the yarns to the first rigid body.

Alternatively, the end of the first rigid body facing the concave surface of the second rigid body may be a wall provided with a hole and the first rigid body is again hollow. The yarns penetrate through the hole into the first rigid body. A knot is formed inside the first rigid body from the yarns. The knot has a larger size than the diameter of the hole so that the yarns 31,32,33,34 are fixed to the first rigid body. The movement of the first rigid body with respect to the second rigid body is dominated by the yarns engaging with the rim of the hole, which rim is considered to be the engaging portion of the first rigid body in this embodiment.

In another variation, the first rigid body may be shaped as a rod provided with a groove on its side wall. The yarns may be wound around the groove to be fixed thereon. The groove serves as the engaging portion in this case.

1. A hinge comprising a first rigid body, a second rigid body and a joint member connected to the first rigid body and the second rigid body, the joint member comprises at least three base connectors, each base connector connected to the second rigid body at a respective connection point and engaging with an engaging portion of the first rigid body, wherein the base connectors are yarns made from fibers and at least a part of the fibers are synthetic organic fibers.

2. The hinge according to claim 1, wherein the at least three base connectors each comprises HPPE fibers, preferably the fibers of the base connectors substantially consist of HPPE fibers.

3. The hinge according to claim 1 further comprising a further connector connected to the second rigid body at a further connection point and engaging with an engaging portion of the first rigid body, the connection points and the further connection point defining a polyhedron within which the engaging portion of the first rigid body is positioned.

4. The hinge according to claim 3, wherein the stiffness of the further connector is lower than the stiffness of the base connectors, preferably the stiffness of the further connector is less than 50% of stiffness of the base connectors.

5. The hinge according to claim 1, wherein the first and/or the second rigid bodies comprise a metal or a composite material.

6. The hinge according to claim 1, wherein the second rigid body has a generally hemispherical shape.

7. The hinge according to claim 1, wherein the first rigid body comprises a rod-shaped portion, which includes the engaging portion of the first body.

8. The hinge according to claim 7, wherein the further connector extends from the engaging portion of the first rigid body to the further connection point in the second rigid body generally in the direction of the axis of the rod-shaped portion in an unloaded state.

9. The hinge according to claim 1, wherein the angles between two base connectors selected from a group consisting of the at least three base connectors are substantially equal in an unloaded state.

10. The hinge according to claim 1, wherein the angles between one of the base connectors and the further connector is substantially equal for all of the base connectors in an unloaded state.

11. The hinge according to claim 1, wherein the first rigid body comprises an elongated portion including the engaging portion, the second rigid body has a generally hemisphere shape and the joint member consists of three base connectors and one further connector, and wherein the angle between any two connectors selected from the four connectors of the joint member is in the range between 100° to 120° in an unloaded state, and preferably about 109.5° in an unloaded state.

12. An apparatus comprising the hinge according to claim 1.

13. A medical device comprising the hinge according to claim 1.

14. An artificial hip joint comprising the hinge as claimed in claim 1, including a femur head part comprising the first rigid body and an acetabulum part comprising the second rigid body.

15. An artificial shoulder joint comprising the hinge as claimed in claim 1, including a humerus head part comprising the first rigid body and a glenoid part comprising the second rigid body.
